(19) United States
(12) Patent Application Publication
(10) Pub. No.: US 2026/0144834 A1
(43) Pub. Date: May 28, 2026

(54) ANTIMICROBIAL FEED ADDITIVE

(71) Applicant: Axitan Ltd., Stevenage (GB)

(72) Inventors: Francisco J. Navarro, Stevenage (GB); Weiluo Lee, Stevenage (GB); Elena Krokidi, Stevenage (GB); Richard Lewis, Stevenage (GB); Kane Miller, Stevenage (GB)

(21) Appl. No.: 18/958,214

(22) Filed: Nov. 25, 2024

(51) Int. Cl. A61K 36/05; A01N 25/00; A01N 37/02; A01N 37/06; A01N 63/50; A01N 65/03; A01P 1/00; A61K 31/20; A61K 31/201; A61K 38/47

(52) U.S. Cl. CPC A61K 36/05; A01N 25/002; A01N 37/02; A01N 37/06; A01N 63/50; A01N 65/03; A01P 1/00; A61K 31/20; A61K 31/201; A61K 38/47

(57) ABSTRACT

Feed additives including Chlamydomonas reinhardtii and a medium-chain fatty acid provide reduced risk of bacterial infection in animals. The feed additives are administered to animals in feed compositions.

### FIELD OF THE DISCLOSURE

The present disclosure is directed toward antimicrobial animal feed additives that include endolysins.

### BACKGROUND

Growing antimicrobial resistance presents unique challenges for all industries, in particular animal health and welfare. New approaches to combat resistance are required to continue to meet growing demand for resources. Where traditional solutions such as antibiotics continue to meet growing resistance, new methods are needed to reduce the risk of infection in animals from feed.

### SUMMARY

Provided herein are feed additive compositions having antimicrobial activity. The feed additive compositions include a Chlamydomonas reinhardtii biomass and a C₆-C₁₄ fatty acid, such as lauric acid. The Chlamydomonas reinhardtii may be present in the form of a dry powder or dry flakes. The Chlamydomonas reinhardtii biomass may be present in an amount from about 25% to about 50% by weight of the feed additive. In particular embodiments, the Chlamydomonas reinhardtii comprises Chlamydomonas reinhardtii strain crAL082. The Chlamydomonas reinhardtii strain crAL082 includes the endolysins AMI2phiZP2 and GH25CPFORC3. In some embodiments, the feed additive composition further includes a long chain fatty acid, such as oleic acid. In some embodiments, the C₆-C₁₄ fatty acid may be present as a desiccated biomass, such as desiccated coconut. In a specific embodiment, the feed additive composition includes a powder of Chlamydomonas reinhardtii strain crAL082; lauric acid; and oleic acid.

Further provided herein are methods for preventing bacterial contamination in a feed. The method includes adding a feed additive to a feed composition, wherein the feed additive includes a Chlamydomonas reinhardtii biomass and a C₆-C₁₄ fatty acid, such as lauric acid. The feed including the feed additive is then administered to the animal.

Further provided herein are methods for preventing bacterial infection in an animal. The methods include administering a feed composition including a feed additive to an animal in need thereof, the feed additive comprising a Chlamydomonas reinhardtii biomass and a C₆-C₁₄ fatty acid, such as lauric acid. The rate of bacterial infection is reduced as compared to an animal administered a feed composition that does not include the feed additive. In some embodiments, the animal is poultry or swine.

### DETAILED DESCRIPTION

Before various aspects of the present invention are disclosed and described, it is to be understood that this invention is not limited to the particular methods, compositions, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity and in another example, a numerical range of “about 50 mg/mL to about 80 mg/mL” should also be understood to provide support for the range of “50 mg/mL to 80 mg/mL.”

In this disclosure, the terms “including,” “containing,” and/or “having” are understood to mean comprising, and are open ended terms.

Described herein are feed additives and feed compositions that include the microalga Chlamydomonas reinhardtii and a C₆-C₁₄ fatty acid. It was surprisingly discovered that combining strains of Chlamydomonas reinhardtii, such as strain crAL082, that express the endolysins AMI2phiZP2 and GH25CPFORC3 with a C₆-C₁₄ fatty acid is effective at reducing the activity of bacteria such as Clostridium perfringens. These endolysins are derived from bacteriophages that infect Clostridium perfringens.

### 1. Feed Additive

The feed additive of the present disclosure includes Chlamydomonas reinhardtii and a C₆-C₁₄ fatty acid. In preferred embodiments, the Chlamydomonas reinhardtii is strain crAL082, which expresses the endolysins AMI2phiZP2 and GH25CPFORC3. This particular strain of Chlamydomonas reinhardtii and the endolysins AMI2phiZP2 and GH25CPFORC3 and methods of making the same are described in WO 2020/157519 A1, the entire contents of which are incorporated by reference herein. The Chlamydomonas reinhardtii may include at least one additional endolysin, which may include endolysin(s) that are produced in other microorganisms.

The Chlamydomonas reinhardtii microalga may be present in the form of a dry powder. For example, when the Chlamydomonas reinhardtii is in the form of a dry powder, the dry powder may be a spray-dried powder, a lyophilized powder, a drum-dried powder, etc. Accordingly, the Chlamydomonas reinhardtii may be formed by spray-drying, lyophilizing (freeze-drying), or drum-drying the Chlamydomonas reinhardtii.

Alternatively, the Chlamydomonas reinhardtii microalga may be present in the form of dry flakes. The dry flakes may be formed by lyophilizing (freeze-drying), drum-drying, or air drying flakes of the Chlamydomonas reinhardtii microalga.

The Chlamydomonas reinhardtii microalga may be present in an amount from about 25% to about 50% by weight of the feed additive. For example, the Chlamydomonas reinhardtii may be present in an amount from about 25% to about 30%, about 25% to about 35%, about 25% to about 40%, about 25% to about 45%, about 25% to about 50%, about 30% to about 50%, about 35% to about 50%, about 40% to about 50%, about 45% to about 50%, or about 30% to about 40% by weight of the feed additive. In some aspects, the Chlamydomonas reinhardtii may be present in an amount of about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or about 50% by weight of the feed additive. In an example, the Chlamydomonas reinhardtii is present in the feed additive in an amount of about 35% by weight.

The C₆-C₁₄ fatty acid may include any C₆-C₁₄ saturated or unsaturated fatty acid. For example, the C₆-C₁₄ fatty acid may be a saturated fatty acid and may include lauric acid, caproic acid, enanthic acid, caprylic acid, pelargonic acid, capric acid, undecylic acid, or any combination thereof. In a preferred embodiment, the C₆-C₁₄ fatty acid includes lauric acid.

The C₆-C₁₄ fatty acid may be present in the feed additive in an amount from about 50% to about 75% by weight of the feed additive. For example, the C₆-C₁₄ fatty acid may be present in the feed additive in an amount from about 50% to about 55%, about 50% to about 60%, about 50% to about 65%, about 50% to about 70%, about 50% to about 75%, about 55% to about 75%, about 60% to about 75%, about 65% to about 75%, about 70% to about 75%, or about 60% to about 70% by weight of the feed additive. In some aspects, the C₆-C₁₄ fatty acid may be present in the feed additive in an amount of about 50%, 51%, 52%, 53%, 54%, 55%, 56%, 57%, 58%, 59%, 60%, 61%, 62%, 63%, 64%, 65%, 66%, 67%, 68%, 69%, or about 70% by weight of the feed additive.

The C₆-C₁₄ fatty acid may be purified or may be included in the feed additive in an unpurified form. For example, the C₆-C₁₄ fatty acid may be included in the form of desiccated biomass, such as desiccated coconut. The desiccated biomass may be present in the feed additive in the form of a dry powder or dry flakes. For example, the desiccated biomass may be formed by spray-drying, lyophilizing, drum drying, or air drying the biomass.

When a desiccated biomass is used to supply the C₆-C₁₄ fatty acid, the desiccated biomass may be present in the feed additive in an amount from about 10% to about 60% by weight, with the balance being the Chlamydomonas reinhardtii biomass and any additional ingredients in the feed additive. For example, the desiccated biomass may be present in the feed additive in an amount from about 10% to about 20%, about 10% to about 30%, about 10% to about 40%, about 10% to about 50%, about 10% to about 60%, about 20% to about 60%, about 30% to about 60%, about 40% to about 60%, about 50% to about 60%, about 20% to about 50%, or about 20% to about 40% by weight of the feed additive. In some aspects, the desiccated biomass may be present in the feed additive in an amount of about 10%, 15%, 20%, 25%, 30%, 35%, 40%, 45%, 50%, 55%, or about 60% by weight of the feed additive.

Generally, the Chlamydomonas reinhardtii and the C₆-C₁₄ fatty acid may be included in a weight ratio from about 1:1 to about 1:3 Chlamydomonas reinhardtii: C₆-C₁₄ fatty acid. For example, the Chlamydomonas reinhardtii and the C₆-C₁₄ fatty acid may be included in a weight ratio from about 1:1 to about 1:1.5, about 1:1 to about 1:2, about 1:1 to about 1:2.5, about 1:1 to about 1:3, about 1:1.5 to about 1:3, about 1:2 to about 1:3, or about 1:2.5 to about 1:3. In some aspects, the Chlamydomonas reinhardtii and the C₆-C₁₄ fatty acid may be included in a weight ratio of about 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, or about 1:3.

When a desiccated biomass is used to supply the C₆-C₁₄ fatty acid, the weight ratio of desiccated biomass to Chlamydomonas reinhardtii biomass may be from 1:4 to about 6:4. For example, the desiccated biomass and the Chlamydomonas reinhardtii biomass may be included in a weight ratio from about 1:4 to about 2:4, about 1:4 to about 3:4, about 1:4 to about 4:4, about 1:4 to about 5:4, about 1:4 to about 6:4, about 2:4 to about 6:4, about 3:4 to about 6:4, about 4:4 to about 6:4, about 5:4 to about 6:4, about 2:4 to about 5:4, or about 3:4 to about 5:4. In some aspects, the desiccated biomass and the Chlamydomonas reinhardtii biomass may be included in a weight ratio of about 1:4, 1.5:4, 2:4, 2.5:4, 3:4, 3.5:4, 4:4, 4.5:4, 5:4, 5.5:4, or about 6:4.

In addition to the C₆-C₁₄ fatty acid, the feed additive may include a short-chain saturated fatty acid, such as propionic acid, butyric acid, valeric acid, or combinations thereof.

Alternatively or in addition to the C₆-C₁₄ fatty acid, the feed additive may include a long-chain or very long-chain saturated or unsaturated fatty acid, such as palmitic acid, stearic acid, oleic acid, linolenic acid, linoleic acid, pinolenic acid, arachidonic acid, eicosapentaenoic acid, docosahexaenoic acid, 4, 7, 10, 13-hexadecatetranoic acid (C16:4), hexadecadienoic acid (C16:2), alpha-linolenic acid (C18:3), or combinations thereof.

Alternatively or in addition to the C₆-C₁₄ fatty acid, the feed additive may include fatty acid derivatives, such as monoglycerides, diglycerides, and triglycerides. For example, the feed additive may include monolinolein or monolaurin.

In some embodiments, the feed additive may further include a monounsaturated long-chain fatty acid. The monounsaturated long-chain fatty acid may include any C₁₃-C₂₁ fatty acid including a single double bond in the carbon chain. For example, the monounsaturated long-chain fatty acid may include oleic acid, palmitoleic acid, vaccenic acid, paullinic acid, elaidic acid, gondoic acid, or combinations thereof. In some preferred embodiments, the mono-unsaturated long-chain fatty acid may include oleic acid, which has high antimicrobial activity and may enhance the antimicrobial activity of the formulation. In particular, the oleic acid may enhance the antibacterial activity of the formulation.

When present, the monounsaturated long-chain fatty acid may be present in an amount from greater than 0% to about 10% by weight of the feed additive. For example, the monounsaturated long-chain fatty acid may be present in an amount from greater than 0% to about 2%, greater than 0% to about 4%, greater than 0% to about 6%, greater than 0% to about 8%, greater than 0% to about 10%, about 2% to about 10%, about 4% to about 10%, about 6% to about 10%, about 8% to about 10%, about 2% to about 8%, or about 4% to about 6% by weight of the feed additive. In some aspects, the monounsaturated long-chain fatty acid may be present in an amount of about 0.5%, 1%, 2%, 3%, 4%, 5%, 6%, 7%, 8%, 9%, or about 10% by weight of the feed additive.

The feed additive may further optionally include one or more excipients. For example, the feed additive may include antioxidants, stabilizers (such as thermal stabilizers), anticaking agents, oil carriers, and the like. The one or more excipients may be present in the feed additive in an amount from about 0.1% to about 10% by weight of the feed additive. For example, the one or more excipients may be present in the feed additive in an amount from about 0.1% to about 0.5%, about 0.1% to about 1%, about 0.1% to about 5%, about 0.1% to about 10%, about 0.5% to about 10%, about 1% to about 10%, about 5% to about 10%, or about 0.5% to about 5% by weight of the feed additive. As another example, the one or more excipients may be present in the feed additive in an amount of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or about 10% by weight of the feed additive.

The excipient may include an antioxidant. The antioxidant may be natural or it may be synthetic. Non-limiting examples of suitable antioxidants include tocopherols (i.e., alpha-, beta-, gamma- and delta-tocopherol), ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline (ethoxyquin), ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, n-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocotrienols (i.e., alpha-, beta-, gamma- and delta-tocotrienols), tyrosol, vanilic acid, 2,6-di-tert-butyl-4-hydroxymethylphenol (i.e., Ionox 100), 2,4-(tris-3′,5′-bi-tert-butyl-4′-hydroxybenzyl)-mesitylene (i.e., Ionox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivatives thereof, vitamin Q10, wheat germ oil, zeaxanthin, or combinations thereof.

The excipient may include a stabilizer. The stabilizer may include guar gum, carrageenan, sorbic acid, potassium sorbate, tocopherol, d-alpha-tocopherol acetate, resveratrol, rosemary oil, erythorbic acid, sodium erythorbate, sodium ascorbate, iso-ascorbic acid, sodium iso-ascorbate, potassium nitrate, ethyl lauroyl arginate, benzoic acid, ascorbyl palmitate, ascorbyl stearate, sulphurous acid, methyl-p-hydroxy benzoate, methyl paraben, potassium bisulphite, potassium lactate, sodium lactate, sodium diacetate, butylated hydroxyanisole (a mixture of 2-tertiarybutyl-4-hydroxyanisole and 3-tertiarybutyl-4-hydroxyanisole), butylated hydroxytoluene (3,5-ditertiarybutyl-4-hydroxytoluene), potassium metabisulphite, propyl-p-hydroxy benzoate, calcium propionate, calcium sorbate, citric acid esters of mono- and diglycerides, dimethyl dicarbonate, natamycin, propyl gallate, potassium sulfate, thyme extract, potassium benzoate, or combinations thereof.

The excipient may include an anticaking agent. The anticaking agent may include diatomaceous earth, bentonite, calcium carbonate, magnesium carbonate, esterified glucomannan, vermiculite, calcium bentonite, calcium montmorillonite, zeolite aluminosilicate, activated carbon, sodium calcium aluminosilicates, or combinations thereof.

The excipient may include an oil carrier. For example, the oil carrier may include diatomaceous earth, silica-based carriers, lignocellulose-based carriers, and combinations thereof.

The Chlamydomonas reinhardtii may be encapsulated in a material which surrounds and protects the endolysin from the effects or heat, digestive enzymes, or pH changes. The Chlamydomonas reinhardtii may be encapsulated by one or more coating layers. For example, the Chlamydomonas reinhardtii may be encapsulated by a thermoprotectant to protect the Chlamydomonas reinhardtii from thermal degradation. Thermoprotectants may include a salt such as Na₂SO₄, glycine, betaine, choline, sorbitol, mannosylglycerate, or a combination thereof.

Alternatively or additionally, the Chlamydomonas reinhardtii may be encapsulated by one or more enteric polymers, such as poly(methacrylic acid-co-methyl methacrylate) or Eudragit (e.g., EUDRAGIT L30 D-55), which may achieve enteric release of the Chlamydomonas reinhardtii.

Alternatively or additionally, the feed additive may be encapsulated by one or more moisture hydrating materials. The moisture hydrating material may be applied as a coating on the Chlamydomonas reinhardtii material using encapsulating or coating apparatuses generally known in the art. The moisture hydrating material may include inorganic salts, sucrose, starch, maltodextrin, polymers, gums, whey, or any combination thereof.

Other encapsulating agents which may be applied to the Chlamydomonas reinhardtii may include inorganic salts, sucrose, starch, maltodextrin, polymers, gums, whey, or any combination thereof. For example, the encapsulating agents may include polyvinyl acetate, trehalose, chitosan, starch, cellulose derivatives, pectin, carrageenan, gum arabic, gelatin, whey protein, or any combination thereof, or other coating ingredients generally known in the art.

The feed additive may have a stability of about 1 week or more, about 2 weeks or more, about 3 weeks or more, about 4 weeks or more, about 1 month or more, about 2 months or more, about 3 months or more, about 4 months or more, about 5 months or more, about 6 months or more, about 9 months or more, or about 12 months or more when stored in a dry, cool environment away from direct sunlight, such as from 15-25° C. and 40% relative humidity or lower.

### II. Feed Composition

Further provided herein are feed compositions that include an animal feed and the feed additive described in Section I above. The feed composition may be suitable for administration to a subject. As used herein, a “subject” may include an animal, such as a domesticated animal or farm animal. Examples of animals for which the feed composition may be suitable for administration include poultry (e.g., chickens, turkeys, ducks, geese, etc.), swine, cattle, horses, deer, sheep, goats, donkeys, mules, ponies, and the like. In some examples, the feed composition may be suitable for administration to poultry or swine. In particular examples, the poultry includes chickens, such as domestic broiler chickens or laying hens, and turkeys. The feed composition may be in the form of pellets, granules, powders, flakes, mash, and the like.

The feed composition may include an animal feed. Examples of animal feeds include grains, forage products, feed meals, feed concentrates, and the like.

Grains may include corn, soybeans, wheat, barley, oats, sorghum, rye, rice, and other grains, and grain meals (e.g., soybean meal).

Forage products are feed ingredients such as vegetative plants in either a fresh (pasture grass or vegetation), dried, or ensiled state and may incidentally include minor proportions of grain (e.g., kernels of corn that remain in harvested corn plant material after harvest). Forage products also include plants that have been harvested and optionally fermented prior to being provided to animals as a part of their diet. Thus, forage products include hay, haylage, and silage. Examples of hay include harvested grass, either indigenous to the location of the ruminants being fed or shipped to the feeding location from a remote location. Non-limiting examples of hay include alfalfa, Bermuda grass, bahia grass, limpo grass, rye grass, wheat grass, fescue, clover, and the like as well as other grass varieties that may be native to the location of the ruminants being provided the ruminant feed ration. Forage products may also include high fiber sources and scrap vegetation products such as green chop, corncobs, plant stalks, and the like.

Feed concentrates are feedstuffs that are high in energy and low in crude fiber. Concentrates may also include a source of one or more ingredients that are used to enhance the nutritional adequacy of a feed supplement mix, such as vitamins and minerals.

The feed composition containing the animal feed may be prepared by combining an amount of the feed additive into the animal feed. The feed additive may be incorporated into the feed composition in an amount from about 0.01% to about 5% by weight of the feed composition. For example, the feed additive may be incorporated into the feed composition in an amount from about 0.01% to about 0.05%, about 0.01% to about 0.1%, about 0.01% to about 0.5%, about 0.01% to about 1%, about 0.01% to about 5%, about 0.05% to about 5%, about 0.1% to about 5%, about 0.5% to about 5%, about 1% to about 5%, about 0.05% to about 1%, or about 0.1% to about 1% by weight of the feed composition. As another example, the feed additive may be incorporated into the feed composition in an amount of about 0.01%, 0.025%, 0.05%, 0.075%, 0.1%, 0.25%, 0.5%, 0.75%, 1%, 2.5%, or about 5% by weight of the feed composition.

The feed composition may include additional feed ingredients in addition to the feed additive described in Section I. Non-limiting examples of additional feed ingredients include vitamins, minerals, amino acids or amino acid analogs, antioxidants, polyunsaturated fatty acids, enzymes, prebiotics, probiotics, postbiotics, herbs, pigments, approved antibiotics, or combinations thereof.

In some embodiments, the feed ingredient may be one or more vitamins. Suitable vitamins include vitamin A, vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid), vitamin B12, vitamin C, vitamin D, vitamin E, vitamin K, other B-complex vitamins (e.g., choline, carnitine, adenine), or combinations thereof. The form of the vitamin may include salts of the vitamin, derivatives of the vitamin, compounds having the same or similar activity of a vitamin, and metabolites of a vitamin.

In further embodiments, the feed ingredient may be one or more amino acids. Non-limiting suitable amino acids include standard amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), non-standard amino acids (e.g., L-DOPA, GABA, 2-aminobutyric acid, and the like), amino acid analogs (e.g., alpha hydroxy analogs), or combinations thereof.

In alternate embodiments, the feed ingredient may be one or more antioxidants. Suitable antioxidants are provided in Section I above.

In still other embodiments, the feed ingredient may be one or more polyunsaturated fatty acids. Suitable polyunsaturated fatty acids (PUFAs) include long chain fatty acids with at least 18 carbon atoms and at least two carbon-carbon double bonds, generally in the cis-configuration. In specific embodiments, the PUFA may be an omega fatty acid. The PUFA may be an omega-3 fatty acid in which the first double bond occurs in the third carbon-carbon bond from the methyl end of the carbon chain (i.e., opposite the carboxyl acid group). Suitable examples of omega-3 fatty acids include all-cis 7,10,13-hexadecatrienoic acid; all-cis-9,12,15-octadecatrienoic acid (alpha-linolenic acid, ALA); all-cis-6,9,12,15,-octadecatetraenoic acid (stearidonic acid); all-cis-8,11,14,17-eicosatetraenoic acid (eicosatetraenoic acid); all-cis-5,8,11,14,17-eicosapentaenoic acid (eicosapentaenoic acid, EPA); all-cis-7,10,13,16,19-docosapentaenoic acid (clupanodonic acid, DPA); all-cis-4,7,10,13,16,19-docosahexaenoic acid (docosahexaenoic acid, DHA); all-cis-4,7,10,13,16,19-docosahexaenoic acid; and all-cis-6,9,12,15,18,21-tetracosenoic acid (nisinic acid). In an alternative embodiment, the PUFA may be an omega-6 fatty acid in which the first double bond occurs in the sixth carbon-carbon bond from the methyl end of the carbon chain. Examples of omega-6 fatty acids include all-cis-9,12-octadecadienoic acid (linoleic acid); all-cis-6,9,12-octadecatrienoic acid (gamma-linolenic acid, GLA); all-cis-11,14-eicosadienoic acid (eicosadienoic acid); all-cis-8,11,14-eicosatrienoic acid (dihomo-gamma-linolenic acid, DGLA); all-cis-5,8,11,14-eicosatetraenoic acid (arachidonic acid, AA); all-cis-13,16-docosadienoic acid (docosadienoic acid); all-cis-7,10,13,16-docosatetraenoic acid (adrenic acid); and all-cis-4,7,10,13,16-docosapentaenoic acid (docosapentaenoic acid). In yet another alternative embodiment, the PUFA may be an omega-9 fatty acid in which the first double bond occurs in the ninth carbon-carbon bond from the methyl end of the carbon chain, or a conjugated fatty acid, in which at least one pair of double bonds are separated by only one single bond. Suitable examples of omega-9 fatty acids include cis-9-octadecenoic acid (oleic acid); cis-11-eicosenoic acid (eicosenoic acid); all-cis-5,8,11-eicosatrienoic acid (mead acid); cis-13-docosenoic acid (erucic acid), and cis-15-tetracosenoic acid (nervonic acid). Examples of conjugated fatty acids include 9Z,11E-octadeca-9,11-dienoic acid (rumenic acid); 10E,12Z-octadeca-9,11-dienoic acid; 8E,10E,12Z-octadecatrienoic acid (a-calendic acid); 8E,10E,12E-octadecatrienoic acid (13-Calendic acid); 8E,10Z,12E-octadecatrienoic acid (jacaric acid); 9E,11E,13Z-octadeca-9,11,13-trienoic acid (a-eleostearic acid); 9E,11E,13E-octadeca-9,11,13-trienoic acid (13-eleostearic acid); 9Z,11Z,13E-octadeca-9,11,13-trienoic acid (catalpic acid), and 9E,11Z,13E-octadeca-9,11,13-trienoic acid (punicic acid).

In still other embodiments, the feed ingredient may be one or more probiotics, prebiotics, or postbiotics. Probiotics, prebiotics, and postbiotics include agents derived from yeast or bacteria that promote good digestive health. By way of non-limiting example, yeast-derived probiotics and prebiotics include yeast cell wall derived components such as β-glucans, arabinoxylan isomaltose, agarooligosaccharides, lactosucrose, cyclodextrins, lactose, fructooligosaccharides, laminariheptaose, lactulose, β-galactooligosaccharides, mannanoligosaccharides, raffinose, stachyose, oligofructose, glucosyl sucrose, sucrose thermal oligosaccharide, isomalturose, caramel, inulin, and xylooligosaccharides. In an exemplary embodiment, the yeast-derived agent may be β-glucans and/or mannanoligosaccharides. Sources for yeast cell wall derived components include Saccharomyces bisporus, Saccharomyces boulardii, Saccharomyces cerevisiae, Saccharomyces capsularis, Saccharomyces delbrueckii, Saccharomyces fermentati, Saccharomyces lugwigii, Saccharomyces microellipsoides, Saccharomyces pastorianus, Saccharomyces rosei, Candida albicans, Candida cloaceae, Candida tropicalis, Candida utilis, Geotrichum candidum, Hansenula americana, Hansenula anomala, Hansenula wingei, and Aspergillus oryzae. Probiotics, prebiotics, and postbiotics may also include bacteria cell wall derived agents such as peptidoglycan and other components derived from gram-positive bacteria with a high content of peptidoglycan. Exemplary gram-positive bacteria include Lactobacillus acidophilus, Bifidobacterium thermophilum, Bifidobacterium longhum, Streptococcus faecium, Bacillus pumilus, Bacillus subtilis, Bacillus licheniformis, Lactobacillus acidophilus, Lactobacillus casei, Enterococcus faecium, Bifidobacterium bifidum, Propionibacterium acidipropionici, Propionibacteriium freudenreichfi, and Bifidobacterium pseudolongum.

In alternate embodiments, the feed ingredient may be one or more enzymes or enzyme variants. Suitable non-limiting examples of enzymes include amylases, carbohydrases, cellulases, esterases, galactonases, galactosidases, glucanases, hemicellulases, hydrolases, lipases, oxidoreductases, pectinases, peptidases, phosphatases, phospholipases, phytases, proteases, transferases, xylanases, or combinations thereof.

In further embodiments, the feed ingredient may be one or more herbals. Suitable herbals and herbal derivatives, as used herein, refer to herbal extracts, and substances derived from plants and plant parts, such as leaves, flowers, and roots, without limitation. Non-limiting exemplary herbals and herbal derivatives include agrimony, alfalfa, aloe vera, amaranth, angelica, anise, barberry, basil, bayberry, bee pollen, birch, bistort, blackberry, black cohosh, black walnut, blessed thistle, blue cohosh, blue vervain, boneset, borage, buchu, buckthorn, bugleweed, burdock, capsicum, cayenne, caraway, cascara sagrada, catnip, celery, centaury, chamomile, chaparral, chickweed, chicory, chinchona, cloves, coltsfoot, comfrey, cornsilk, couch grass, cramp bark, culver's root, cyani, cornflower, damiana, dandelion, devils claw, dong quai, echinacea, elecampane, ephedra, eucalyptus, evening primrose, eyebright, false unicorn, fennel, fenugreek, figwort, flaxseed, garlic, gentian, ginger, ginseng, golden seal, gotu kola, gum weed, hawthorn, hops, horehound, horseradish, horsetail, hoshouwu, hydrangea, hyssop, iceland moss, irish moss, jojoba, juniper, kelp, lady's slipper, lemon grass, licorice, lobelia, mandrake, marigold, marjoram, marshmallow, mistletoe, mullein, mustard, myrrh, nettle, oatstraw, oregon grape, papaya, parsley, passion flower, peach, pennyroyal, peppermint, periwinkle, plantain, pleurisy root, pokeweed, prickly ash, psyllium, quassia, queen of the meadow, red clover, red raspberry, redmond clay, rhubarb, rose hips, rosemary, rue, safflower, saffron, sage, St. John's wort, sarsaparilla, sassafras, saw palmetto, scullcap, senega, senna, shepherd's purse, slippery elm, spearmint, spikenard, squawvine, stillingia, strawberry, taheebo, thyme, uva ursi, valerian, violet, watercress, white oak bark, white pine bark, wild cherry, wild lettuce, wild yam, willow, wintergreen, witch hazel, wood betony, wormwood, yarrow, yellow dock, yerba santa, yucca, or combinations thereof.

In still other embodiments, the feed ingredient may be one or more natural pigments. Suitable pigments include, without limit, actinioerythrin, alizarin, alloxanthin, β-apo-2′-carotenal, apo-2-lycopenal, apo-6′-lycopenal, astacein, astaxanthin, azafrinaldehyde, aacterioruberin, aixin, α-carotene, β-carotene, γ-carotene, β-carotenone, canthaxanthin, capsanthin, capsorubin, citranaxanthin, citroxanthin, crocetin, crocetinsemialdehyde, crocin, crustaxanthin, cryptocapsin, α-cryptoxanthin, β-cryptoxanthin, cryptomonaxanthin, cynthiaxanthin, decaprenoxanthin, dehydroadonirubin, diadinoxanthin, 1,4-diamino-2,3-dihydroanthraquinone, 1,4-dihydroxyanthraquinone, 2,2′-diketospirilloxanthin, eschscholtzxanthin, eschscholtzxanthone, flexixanthin, foliachrome, fucoxanthin, gazaniaxanthin, hexahydrolycopene, hopkinsiaxanthin, hydroxyspheriodenone, isofucoxanthin, loroxanthin, lutein, luteoxanthin, lycopene, lycopersene, lycoxanthin, morindone, mutatoxanthin, neochrome, neoxanthin, nonaprenoxanthin, OH-Chlorobactene, okenone, oscillaxanthin, paracentrone, pectenolone, pectenoxanthin, peridinin, phleixanthophyll, phoeniconone, phoenicopterone, phoenicoxanthin, physalien, phytofluene, pyrrhoxanthininol, quinones, rhodopin, rhodopinal, rhodopinol, rhodovibrin, rhodoxanthin, rubixanthone, saproxanthin, semi-α-carotenone, semi-β-carotenone, sintaxanthin, siphonaxanthin, siphonein, spheroidene, tangeraxanthin, torularhodin, torularhodin methyl ester, torularhodinaldehyde, torulene, 1,2,4-trihydroxyanthraquinone, triphasiaxanthin, trollichrome, vaucheriaxanthin, violaxanthin, warn ingone, xanthin, zeaxanthin, α-zeacarotene, or combinations thereof.

In yet other embodiments, the feed ingredient may be one or more antibiotics approved for use in livestock and poultry (i.e., antibiotics not considered critical or important for human health). Non-limiting examples of approved antibiotics include bacitracin, carbadox, ceftiofur, enrofloxacin, florfenicol, laidlomycin, linomycin, oxytetracycline, roxarsone, tilmicosin, tylosin, and virginiamycin.

In further embodiments, the feed ingredient may be a protein meal (e.g., soybean meal, corn meal, corn gluten meal, corn germ meal, fish meal, blood meal, bone meal, poultry by-product meal, etc.), protein cakes (e.g., soybean cake, soybean oil cake, and so forth), grains (e.g., corn grain, oat grain, wheat grain, milled grains, brewer's grains, sprouted grains, and so forth), pelleted feeds, oils or fats (e.g., vegetable oils, oilseed oils, rendered animal fats, and so forth).

### III. Methods of Use

Further provided herein are methods for inhibiting or preventing bacterial infection in an animal. As used herein “inhibiting” or “preventing” bacterial infection may refer to a reduction in the risk that an animal consumes a feed containing the pathogenic bacteria, or it may refer to a reduction in the likelihood that an animal that consumes the pathogenic bacteria shows symptoms of infection.

The bacterial infection may be caused by any Clostridium bacterial species, such as Clostridium perfringens, Clostridium septicum, Clostridium botulinum, Clostridium tetani, and others known in the art. In some examples, the bacterial infection may be caused by Clostridium perfringens.

The methods generally include administering a feed additive as described in Section I above to the animal. The feed additive may be incorporated into a feed composition as described in Section II when administered to the animal.

Further provided herein are methods for preventing bacterial contamination in an animal feed. The method generally comprises combining an animal feed with a feed additive described in Section I. The methods may further include administering the feed to an animal.

The feed additive may be administered to an animal in need thereof by adding the feed additive to a feed composition as described in Section II above. The feed additive may be added to the feed composition immediately before administering the feed composition to the animal, Alternatively, the feed additive may be added to the feed composition at an earlier time during processing of the feed composition, as described more detail below.

The feed additive may be administered to the animal by methods and apparatuses known to those having ordinary skill in the art. For example, the feed composition may be added to a feeding container such as a bin or trough suitable for the animal to feed from.

### IV. Processes for Making

Further provided herein are processes for making the feed additives described in Section I above. The processes generally include combining Chlamydomonas reinhardtii as described in Section I with a C₆-C₁₄ fatty acid. The Chlamydomonas reinhardtii may be cultured according to the methods described in WO 2020/157519 A1, the entire contents of which are incorporated by reference herein in their entirety. Once cultured, the Chlamydomonas reinhardtii may be dried by spray-drying, lyophilizing (freeze-drying), or drum-drying the culture. The Chlamydomonas reinhardtii may then be combined with the C₆-C₁₄ fatty acid and any other ingredients described in Section I by mixing. The mixing may be accomplished by any mixing apparatus known in the art, such as a vertical blender or ribbon mixer.

Further provided herein are processes for making the feed compositions described in Section II above. The processes generally include combining the feed additives with the animal feed. This may be done immediately prior to administering the feed to an animal, or it may be done during production of the feed composition.

Preferably the feed composition is a pelleted feed. Such feed is commonly manufactured by (i) crushing of the raw materials (i.e., the animal feed and any additional feed ingredients), (ii) batching of the raw materials, where the crushed materials/ingredients are measured and mixed in specific proportions to ensure consistency between batches of feed, (iii), mixing, where the ingredients are thoroughly mixed and blended using a blending machine to ensure even distribution of the ingredients throughout the mixture, (iv) conditioning, where the mixture is conditioned using steam and heat, followed by (v) pelleting where the steamed/heated mixture is compressed into compact pellets by extrusion of the mixture through a perforated die plate. Preferably, the feed additive may be added to the other feed ingredients at the batching stage, prior to conditioning and pelleting. Alternatively, the feed additive may be added to the feed as a top dressing after pelleting.

The crushing of step (i) may be accomplished using crushing equipment known to crush the raw materials into a powder. Such crushing equipment is known to those having ordinary skill in the art.

The conditioning of step (iv) may be accomplished at a temperature from about 50° C. to about 100° C., such as from about 70° C. to about 100° C., or about 90° C. to about 100° C. The conditioning step may take place for a period of time from about 30 seconds to about 10 minutes, such as from about 1 minute to about 5 minutes, or about 1 minute to about 2 minutes.

The pelleting of step (v) may be accomplished by any pelleting or extrusion device known in the art. Once the pellets are formed, the pellets may be allowed to cool to a temperature of about 30° C. to about 50° C. before further processing, such as packaging.

### ENUMERATED EMBODIMENTS

Embodiment 1: A feed additive composition comprising: Chlamydomonas reinhardtii; and a C₆-C₁₄ fatty acid.

Embodiment 2: The feed additive of embodiment 1, wherein the Chlamydomonas reinhardtii is present in the form of a dry powder or dry flakes.

Embodiment 3: The feed additive of embodiment 2, wherein the dry powder is a spray-dried powder, a lyophilized powder, or a drum-dried powder.

Embodiment 4: The feed additive of any one of embodiments 1-3, wherein the C₆-C₁₄ fatty acid comprises lauric acid.

Embodiment 5: The feed additive of any one of embodiments 1-4, wherein the Chlamydomonas reinhardtii is present in an amount from about 25% to about 50% by weight of the feed additive.

Embodiment 6: The feed additive of embodiment 5, wherein the Chlamydomonas reinhardtii is present in an amount of about 35% by weight of the feed additive.

Embodiment 7: The feed additive of any one of embodiments 1-6, wherein the Chlamydomonas reinhardtii comprises Chlamydomonas reinhardtii strain crAL082.

Embodiment 8: The feed additive of embodiment 4, wherein the lauric acid is present in an amount from about 50% to about 75% by weight of the feed additive.

Embodiment 9: The feed additive of embodiment 8, wherein the lauric acid is present in an amount of about 60% by weight of the feed additive.

Embodiment 10: The feed additive of any one of embodiments 1-9, further comprising an unsaturated long chain fatty acid.

Embodiment 11: The feed additive of embodiment 10, wherein the unsaturated long chain fatty acid comprises oleic acid.

Embodiment 12: The feed additive of embodiment 11, wherein the oleic acid is present in an amount from greater than 0% to about 10% by weight of the feed additive.

Embodiment 13: The feed additive of embodiment 11, wherein the oleic acid is present in an amount of about 5% by weight of the feed additive.

Embodiment 14: The feed additive of embodiment 7, wherein the Chlamydomonas reinhardtii strain crAL082 comprises the endolysins AM12phiZP2 and GH25CPFORC3.

Embodiment 15: A feed additive composition consisting of: a powder of Chlamydomonas reinhardtii strain crAL082; lauric acid; and oleic acid.

Embodiment 16: The feed additive of embodiment 15, wherein the powder of Chlamydomonas reinhardtii strain crAL082 is present in an amount from about 25% to about 45% by weight of the feed additive.

Embodiment 17: The feed additive of embodiment 16, wherein the powder of Chlamydomonas reinhardtii strain crAL082 is present in an amount of about 35% by weight of the feed additive.

Embodiment 18: The feed additive of any one of embodiments 15-17, wherein the lauric acid is present in an amount from about 50% to about 75% by weight of the feed additive.

Embodiment 19: The feed additive of embodiment 18, wherein the lauric acid is present in an amount of about 60% by weight of the feed additive.

Embodiment 20: The feed additive of any one of embodiments 15-19, wherein the oleic acid is present in an amount from about 1% to about 10% by weight of the feed additive.

Embodiment 21: The feed additive of embodiment 20, wherein the oleic acid is present in an amount of about 5% by weight of the feed additive.

Embodiment 22: A feed additive composition comprising: a powder of Chlamydomonas reinhardtii strain crAL082; desiccated coconut; and oleic acid.

Embodiment 23: A feed additive composition comprising: a powder including the endolysins AM12phiZP2 and GH25CPFORC3; lauric acid; and oleic acid.

Embodiment 24: A feed composition comprising the feed additive of any one of embodiments 1-23.

Embodiment 25: The feed composition of embodiment 24, wherein the feed composition is suitable for administration to poultry or swine.

Embodiment 26: The feed composition of embodiment 25, wherein the feed composition is suitable for administration to broilers, laying hens, turkeys, or a combination thereof.

Embodiment 27: A method for reducing preventing bacterial contamination in a feed, the method comprising adding a feed additive to an animal feed composition, the feed additive comprising: Chlamydomonas reinhardtii; and a C₆-C₁₄ fatty acid.

Embodiment 28: The method of embodiment 27, further comprising administering the feed to an animal.

Embodiment 29: A method for preventing bacterial infection in an animal the method comprising administering a feed composition including a feed additive to an animal in need thereof, the feed additive comprising: Chlamydomonas reinhardtii; and a C₆-C₁₄ fatty acid.

Embodiment 30: The method of embodiment 29, wherein the rate of bacterial infection is reduced as compared to an animal administered a feed composition that does not include the feed additive.

Embodiment 31: The method of embodiment 29 or embodiment 30, wherein the animal is poultry or swine.

Embodiment 32: The method of embodiment 31, wherein the animal is a broiler chicken, laying hen, or turkey.

Embodiment 33: A method for administering a feed additive to an animal comprising adding the feed additive of any one of embodiments 1-23 to a feed composition prior to administering the feed composition to the animal.

Embodiment 34: The method of embodiment 33, wherein the animal is poultry or swine.

Embodiment 35: The method of embodiment 34, wherein the animal is a broiler chicken, laying hen, or turkey.

Embodiment 36: The method of any one of embodiments 33-35, wherein the feed additive is added to the feed composition in an amount from about 0.01% to about 5% by weight of the feed composition.

### EXAMPLES

Examples have been set forth below for the purpose of illustration and to describe certain specific embodiments of the disclosure. However, the scope of the claims is not to be in any way limited by the examples set forth herein. Various changes and modifications to the disclosed embodiments will be apparent to those skilled in the art and such changes and modifications including, without limitation, those relating to the chemical structures, substituents, derivatives, formulations, or methods of the disclosure may be made without departing from the disclosure and the scope of the appended claims. Definitions of the variables in the structures in the schemes herein are commensurate with those of corresponding positions in the formulae presented herein.

### Example 1: Synergy Between Microalgal Extracts Carrying Either GH25CPFORC3 or AM12phiZP2 and Lauric Acid

The interaction between lauric acid and microalgal biomasses containing endolysins GH25CPFORC3 and AM12phiZP2 was determined by comparing the MIC (Minimal inhibitory concentration) values of two compounds tested separately with the MIC of combination of the two compounds at varying ratios. Synergy is defined when the fractional inhibitory concentration index (ΣF) is lower than 0.5. ΣF is the sum of FICa+FICb, where FICa and FICb are the MICs of the compounds in the combination divided by the MICs of the each compound alone.

Lauric acid was first ground in a mortar to a fine powder and then resuspended at 25 mg/ml in phosphate buffered saline at pH 7.0. The suspension was then sonicated until a white homogenous suspension was formed.

Extracts of microalgal biomass were prepared from spray dried powder, resuspended in phosphate buffered saline at approximately 300 mg/ml and homogenized by repeated cycles of freeze and thawing and 25 cycles of sonication at 10 μm amplitude, 5 sec ON, 5 sec OFF. The lysate was then clarified by centrifugation at >31,000×g for 30 min at 20° C. The soluble protein concentration of the clarified lysates was determined using the Biorad DC protein concentration kit.

Bacteria (Clostridium perfringens) were grown overnight under anaerobic conditions at 37° C. in 10 mL BHI+C media (Brain Heart Infusion media supplemented with Cysteine) and then diluted 1:1000 in fresh media for the MIC assay. 90 μl of the diluted bacterial culture were dispensed in wells of a 96-well microtiter plate, together with 10 μl of testing samples. Microtiter plates were covered with aluminum seals and kept shaking for ˜16 h at 41° C. inside the anaerobic chamber. After incubation the turbidity of each well was measured at 600 nm. The MIC value was defined as the minimal concentration of the testing substances that completely inhibited bacteria growth.

The results are shown in Tables 1 and 2. Table 1 shows results of the combination between lauric acid and microalgal extract containing endolysin GH25CPFORC3, and Table 2 shows results of the combination between lauric acid and microalgal extract containing endolysin AM12phiZP2. ΣF values below 0.5 indicate synergy.

TABLE 1

| | | | | microalgal | MIC of | |
|---|---|---|---|---|---|---|
| | | Cell | Lauric | biomass | blend Lauric | |
| | Bacteria | loading | acid | GH25CPFORC3 | acid:biomass | |
| Experiment | strain | (CFU/ml) | (μg/ml) | (μg/ml) | (1:1) | ΣFIC |
| 1 | NCTC | 1.5 × 10⁴ | 185 | 124 | 134 | 0.8 |
| | 2837 | | | | | |
| 2 | NCTC | 1.5 × 10⁵ | 35 | 329 | 50 | 1 |
| | 8238 | | | | | |
| 3 | NCTC | 2.5 × 10⁵ | 260 | 1317 | 141 | 0.4 |
| | 8237 | | | | | |
| 4 | NCTC | 3.9 × 10⁵ | 824 | 173 | 94 | 0.2 |
| | 10578 | | | | | |

TABLE 2

| | | | | microalgal | MIC of | |
|---|---|---|---|---|---|---|
| | | | Lauric | biomass | blend Lauric | |
| | Cp | Cell loading | acid | AMI2phiZP2 | acid:biomass | |
| Experiment | strain | (CFU/ml) | (μg/ml) | (μg/ml) | (1:1) | ΣFIC |
| 1 | NCTC | 1.5 × 10⁴ | 185 | 308 | 232 | 1.1 |
| | 2837 | | | | | |
| 2 | NCTC | 1.5 × 10⁵ | 35 | 370 | 116 | 2.1 |
| | 8238 | | | | | |
| 3 | NCTC | 2.5 × 10⁵ | 260 | 877 | 243 | 0.7 |
| | 8237 | | | | | |
| 4 | NCTC | 3.9 × 10⁵ | 824 | 260 | 126 | 0.3 |
| | 10578 | | | | | |

### Example 2: Collection of MIC Values for Different Fatty Acids Against Clostridium perfringens

Stock solutions of fatty acids were prepared in ethanol and then dispersed at low concentrations in PBS just before the MIC assay. Bacteria (Clostridium perfringens) was refreshed from frozen aliquots prepared from exponential growing cells and diluted in fresh BHI+C media (Brain Heart Infusion media supplemented with Cysteine). 90 μl aliquots of the diluted bacterial culture were dispensed in wells of a 96-well microtiter plate, together with 10 μl of a serial dilution of test samples. Microtiter plates were covered with aluminum seals and kept shaking for ˜16 h at 37° C. inside the anaerobic chamber. After incubation, the turbidity of each well was measured at λ=600 nm. The MIC value was defined as the minimal concentration of the test substances that completely inhibited bacteria growth. The MBC (minimum bactericidal concentration) value was defined as the minimal concentration of the test substances that killed Clostridium perfringens. Results are shown in Table 3.

TABLE 3

| | | | MIC | MBC |
|---|---|---|---|---|
| Fatty acid name | Formula | Strain, CFU/mL | (mg/ml) | (mg/ml) |
| Propionic acid (C3:0) | C3:0 | CL15, 8.1 × 10² CFU/mL | 4.95 | 4.95 |
| Butyric acid (C4:0) | C4:0 | CL15, 1.2 × 10³ CFU/mL | 0.3 | 0.3 |
| Valeric acid (C5:0) | C5:0 | CL15, 8.1 × 10² CFU/mL | 4.8 | 4.8 |
| Pelargonic acid (C9:0) | C9:0 | CL15, 1.7 × 10³ CFU/mL | >0.3 | >0.3 |
| Capric acid (C10:0) | C10:0 | CL15, 1.7 × 10³ CFU/mL | >0.3 | >0.3 |
| Lauric acid (C12:0) | C12:0 | CL15, 1.3 × 10³ CFU/mL | 0.019 | ND |
| Monolaurin (lauryl monoglyceride) | | CL15, 1.2 × 10³ CFU/mL | 0.0375 | 0.0375 |
| Palmitic acid (C16:0) | C16:0 | CL15, 1.4 × 10³ CFU/mL | 0.113 | 0.15 |
| Stearic acid (C18:0) | C18:0 | CL15, 1.8 × 10³ CFU/mL | >0.1 | >0.1 |
| Oleic acid (C18:1) | C18:1 | CL15, 1.4 × 10³ CFU/mL | 0.0084 | 0.0084 |
| Linolenic acid (C18:3) | C18:3 | CL15, 1.2 × 10³ CFU/mL | 0.009 | 0.009 |
| Monolinolein | | CL15, 1.2 × 10³ CFU/mL | 0.01875 | 0.01875 |
| Pinolenic acid (C18:3) | C18:3 | CL15, 1.8 × 10³ CFU/mL | 0.094 | 0.094 |
| AA (C20:4) | C20:4 | CL15, 1.2 × 10³ CFU/mL | 0.005 | 0.005 |
| EPA (C20:5) | C20:5 | CL15, 1.2 × 10³ CFU/mL | 0.012 | 0.012 |
| DHA (C22:6) | C22:6 | CL15, 1.2 × 10³ CFU/mL | 0.005 | 0.003 |

What is claimed is:

1. A feed additive composition comprising:
Chlamydomonas reinhardtii; and
a C₆-C₁₄ fatty acid.

2. The feed additive of claim 1, wherein the Chlamydomonas reinhardtii is present in the form of a dry powder or dry flakes.

3. The feed additive of claim 2, wherein the dry powder is a spray-dried powder, a lyophilized powder, or a drum-dried powder.

4. The feed additive of claim 1, wherein the C₆-C₁₄ fatty acid comprises lauric acid.

5. The feed additive of claim 1, wherein the Chlamydomonas reinhardtii is present in an amount from about 25% to about 50% by weight of the feed additive.

6. The feed additive of claim 5, wherein the Chlamydomonas reinhardtii is present in an amount of about 35% by weight of the feed additive.

7. The feed additive of claim 1, wherein the Chlamydomonas reinhardtii comprises Chlamydomonas reinhardtii strain crAL082.

8. The feed additive of claim 4, wherein the lauric acid is present in an amount from about 50% to about 75% by weight of the feed additive.

9. The feed additive of claim 8, wherein the lauric acid is present in an amount of about 60% by weight of the feed additive.

10. The feed additive of claim 1, further comprising an unsaturated long chain fatty acid.

11. The feed additive of claim 10, wherein the unsaturated long chain fatty acid comprises oleic acid.

12. The feed additive of claim 11, wherein the oleic acid is present in an amount from greater than 0% to about 10% by weight of the feed additive.

13. The feed additive of claim 11, wherein the oleic acid is present in an amount of about 5% by weight of the feed additive.

14. The feed additive of claim 7, wherein the Chlamydomonas reinhardtii strain crAL082 comprises the endolysins AMI2phiZP2 and GH25CPFORC3.

15. The feed additive composition of claim 7, further comprising:
lauric acid; and
oleic acid.

16. A feed composition comprising the feed additive of claim 1.

17. The feed composition of claim 16, wherein the feed composition is suitable for administration to poultry or swine.

18. The feed composition of claim 17, wherein the feed composition is suitable for administration to broilers, laying hens, turkeys, or a combination thereof.

19. A method for preventing bacterial contamination in a feed, the method comprising adding a feed additive to an animal feed composition, the feed additive comprising:
Chlamydomonas reinhardtii; and
a C₆-C₁₄ fatty acid.

20. The method of claim 19, further comprising administering the feed to an animal.

21. A method for preventing bacterial infection in an animal, the method comprising administering a feed composition including a feed additive to an animal in need thereof, the feed additive comprising:
Chlamydomonas reinhardtii; and
a C₆-C₁₄ fatty acid.

22. The method of claim 21, wherein the animal is poultry or swine.

23. The method of claim 22, wherein the animal is a broiler chicken, laying hen, or turkey.
